(19) United States
(12) Patent Application Publication
(10) Pub. No.: US 2025/0032368 A1
(43) Pub. Date: Jan. 30, 2025

(54) PLANT OIL PREPARATION AND PREPARATION METHOD THEREFOR

(71) Applicant: DYNABIO BIOMED TECHNOLOGY CO., LTD., Weihai City (CN)

(72) Inventors: Cun-Zhao Li, Changhua County (TW); Syu-Ming Lai, Taichung City (TW); Ping-Shan Lai, Taichung City (TW)

(21) Appl. No.: 18/717,238

(22) PCT Filed: Dec. 16, 2021
(86) PCT No.: PCT/CN2021/138602

§ 371 Date: Jun. 6, 2024

(51) Int. Cl. A61K 8/02

(52) U.S. Cl. CPC A61K 8/0241; A61K 2800/10; A61K 2800/412; A61K 2800/56; A61K 2800/624; A61K 2800/63; A61K 2800/652

(57) ABSTRACT

A plant oil preparation includes a plurality of particles, each of which including a core layer and at least one protective layer, the core layer including the plant oil, the protective layer encapsulating the core layer and including a phospholipid material and a surfactant; and a carrier, the plurality of particles being dispersed within the carrier. A preparation method for the plant oil preparation includes steps of: (1) mixing the phospholipid material in an aqueous phase to form a dispersion solution; (2) mixing the plant oil in the dispersion solution to encapsulate the plant oil by the phospholipid material and form a plurality of intermediate particles; (3) adding the surfactant, allowing the surfactant to attach to the plurality of intermediate particles and form the plurality of particles; and (4) adding the carrier and dispersing the plurality of particles within the carrier.

### BACKGROUND OF THE INVENTION

### Field of the Invention

The present invention relates to a plant oil preparation and a preparation method therefor.

### Description of the Prior Art

Generally, plant oils are fats and oils obtained from seeds, fruits, branches and leaves of plants. The plant oils are rich in linolenic acid, unsaturated fatty acids and multiple vitamins, and the plant oils may be used as edible oils for health care, or skin care products for moisturizing, anti-wrinkle and other beauty effects. For example, sea buckthorn fruit oil and sea buckthorn seed oil are rich in a variety of organic acids, unsaturated fatty acids, flavonoids, vitamins, superoxide dismutase (SOD) and other active ingredients, which may be used as health supplements to achieve anti-inflammation and antioxidant effects and promote metabolism. Hemp oil contains cannabidiol (CBD) and provides anti-inflammation, anti-anxiety and antioxidant effects. Sacha inchi oil contains unsaturated fatty acids (such as essential fatty acids including linolenic acid and linoleic acid), and provides anti-inflammation effect and is beneficial to skin health and cardiovascular health.

However, the plant oils have poor water solubility, which is not convenient for processing and limits their application scope. When the plant oils are used as skin care products, the skin care products may be difficult to preserve and prone to oil-water separation and deterioration, which results in poor antioxidant effect and stability. When the plant oils are used as health supplements, the health supplements also have problems of poor taste and rancid odour.

The present invention is, therefore, arisen to obviate or at least mitigate the above-mentioned disadvantages.

### SUMMARY OF THE INVENTION

The main object of the present invention is to provide a plant oil preparation and a preparation method therefor, and the plant oil preparation has good water solubility and antioxidant effect.

To achieve the above and other objects, the present invention provides a plant oil preparation, including: a plurality of particles, each of the plurality of particles including a core layer and at least one protective layer, the core layer including the plant oil, the at least one protective layer encapsulating the core layer and including at least one phospholipid material and at least one surfactant; and a carrier, the plurality of particles being dispersed within the carrier.

To achieve the above and other objects, the present invention further provides a preparation method for the plant oil preparation as described above, including following steps of: (1) mixing the at least one phospholipid material in an aqueous phase to form a dispersion solution; (2) mixing the plant oil in the dispersion solution to encapsulate the plant oil by the at least one phospholipid material and form a plurality of intermediate particles; (3) adding the at least one surfactant, allowing the at least one surfactant to attach to the plurality of intermediate particles and form the plurality of particles; and (4) adding the carrier and dispersing the plurality of particles within the carrier.

Specifically, the at least one protective layer includes a first protective layer and a second protective layer, the at least one phospholipid material is disposed on an outer surface of the core layer to form the first protective layer, and the at least one surfactant is disposed on an outer surface of the first protective layer to form the second protective layer.

Specifically, a weight ratio of the at least one phospholipid material to the at least one surfactant is from 1:3 to 1:6.

Specifically, the plant oil is present in an amount of at least 10% by weight of the plant oil preparation.

Specifically, an encapsulation efficiency of the plant oil is larger than or equal to 80%.

Specifically, an effective average particle size of the plurality of particles is between 150 nm and 400 nm.

Specifically, the plant oil includes at least one of sea buckthorn fruit oil, sea buckthorn seed oil, hemp oil and sacha inchi oil.

Specifically, the at least one phospholipid material includes at least one of soybean phospholipid, lecithin, dipalmitoylphosphatidylcholine (DPPC), distearoylphosphatidylcholine (DSPC) and egg phosphatidylcholine (EPC).

Specifically, the at least one surfactant includes at least one of vitamin E-TPGS, polyethylene glycol (PEG), ethoxylated hydrogenated castor oil (RH-40), Tween series surfactant and Span series surfactant.

Specifically, the carrier includes at least one of cyclodextrin, maltodextrin, starch, silicon dioxide, cellulose, hydrogel and silicone hydrogel.

To achieve the above and other objects, the present invention further provides a preparation method for the plant oil preparation as described above, including following steps of:
(1) mixing the at least one phospholipid material in an aqueous phase to form a dispersion solution;
(2) mixing the plant oil in the dispersion solution to encapsulate the plant oil by the at least one phospholipid material and form a plurality of intermediate particles;
(3) adding the at least one surfactant, allowing the at least one surfactant to attach to the plurality of intermediate particles and form the plurality of particles; and
(4) adding the carrier and dispersing the plurality of particles within the carrier.

Specifically, in the step (1), a weight ratio of the at least one phospholipid material to the aqueous phase is from 1:5 to 1:15.

Specifically, in the step (2), a weight ratio of the plant oil to the aqueous phase is from 1:4 to 1:8.

Specifically, the step (1) is performed at a temperature of 70° C. to 90° C., and the steps (2) to (4) are performed at a temperature lower than 70° C.

Specifically, in the steps (1) to (4), a stirring speed maintains in a range of 100 rpm to 300 rpm.

Specifically, in the step (2), the dispersion solution with the plant oil is stirred for no less than 20 minutes.

Specifically, after the step (4), the aqueous phase is removed to obtain the plant oil preparation being solidified.

Specifically, the plant oil preparation being solidified is ground to a size range of 40 mesh to 80 mesh.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a schematic diagram of a preferable embodiment of the present invention;

FIG. 2 is a schematic diagram showing a preparation method according to a preferable embodiment of the present invention; and

FIG. 3 is a block diagram showing the preparation method according to a preferable embodiment of the present invention.

### DETAILED DESCRIPTION OF THE PREFERRED EMBODIMENTS

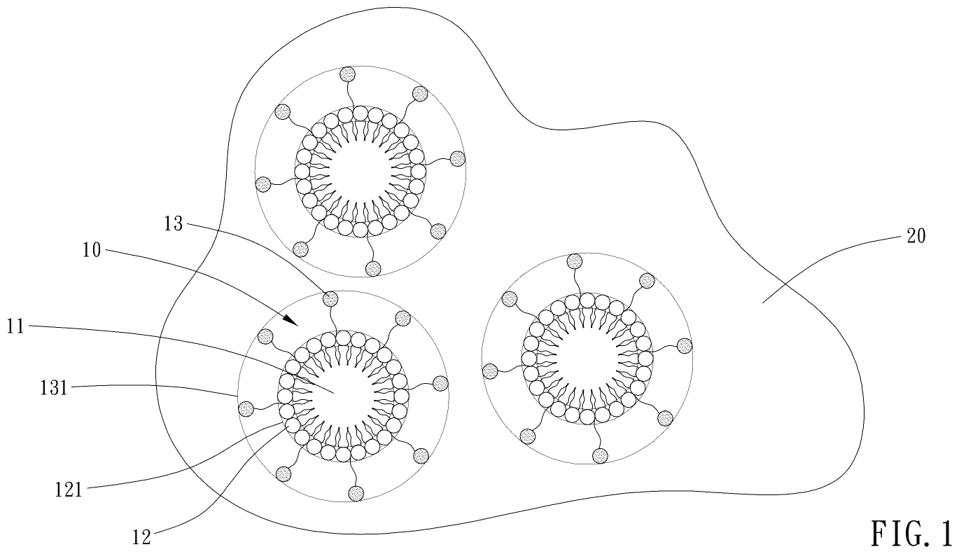

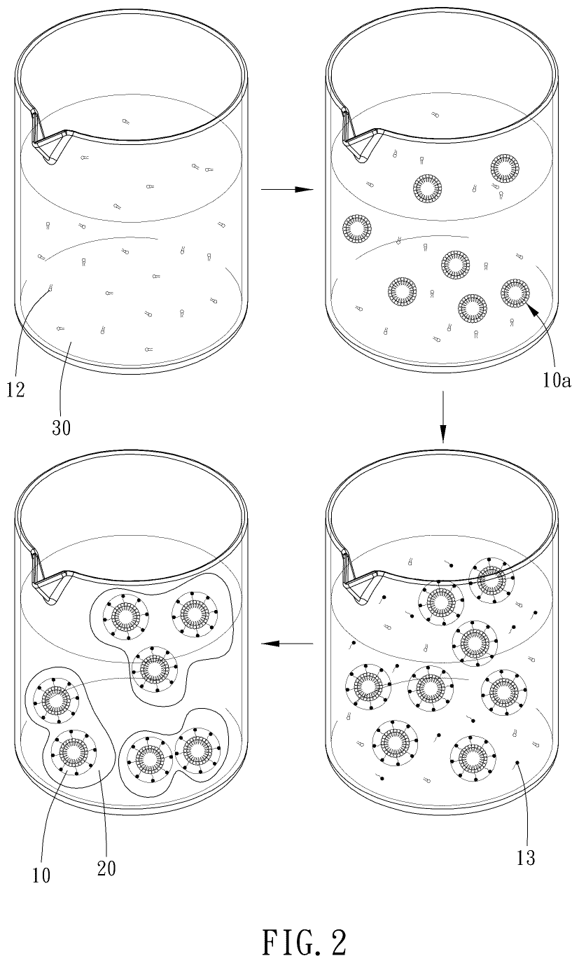

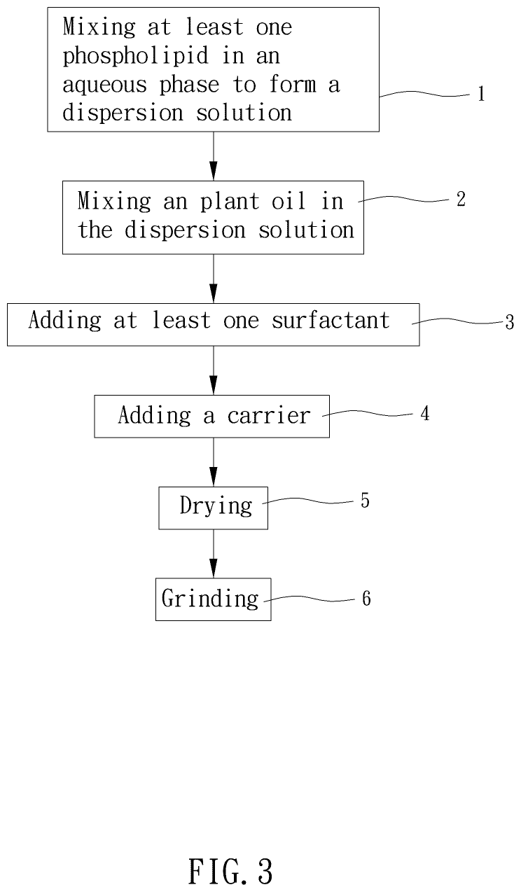

Please refer to FIGS. 1 to 3 for a preferable embodiment of the present invention. A plant oil preparation of the present invention includes a plurality of particles 10 and a carrier 20.

Each of the plurality of particles 10 includes a core layer 11 and at least one protective layer, and the core layer 11 includes the plant oil. The at least one protective layer encapsulates the core layer 11 and includes at least one phospholipid material 12 and at least one surfactant 13. The plurality of particles 10 are dispersed within the carrier 20, which effectively improves the water solubility and antioxidant effect of the plant oil and avoids deterioration for easy storage.

The at least one protective layer includes a first protective layer 121 and second protective layer 131. The at least one phospholipid material 12 is disposed on an outer surface of the core layer 11 to form the first protective layer 121, and the at least one surfactant 13 is disposed on an outer surface of the first protective layer 121 to form the second protective layer 131 so that the plant oil is stably dispersed within the carrier 20 and not easy to aggregate and result in oil-water separation. An effective average particle size of the plurality of particles 10 is between 150 nm and 400 nm, which is easily utilized by human body. Wherein the effective average particle size refers to that, at least about 50% of the plurality of particles 10 have the effective average particle size when measured by a dynamic light scattering (DLS) machine.

The plant oil includes at least one of sea buckthorn fruit oil, sea buckthorn seed oil, hemp oil and sacha inchi oil. Preferably, the hemp oil refers to those containing cannabidiol (CBD). An encapsulation efficiency (EE) of the plant oil is larger than or equal to 80%; the plant oil is present in an amount of at least 10% by weight of the plant oil preparation so as to have sufficient effective active ingredients.

A weight ratio of the at least one phospholipid material 12 to the at least one surfactant 13 is from 1:3 to 1:6, which allows the at least one surfactant 13 to be evenly attached to the first protective layer 121. The at least one phospholipid material 12 includes at least one of soybean phospholipid, lecithin, dipalmitoylphosphatidylcholine (DPPC), distearoylphosphatidylcholine (DSPC) and egg phosphatidylcholine (EPC). The at least one surfactant 13 includes at least one of vitamin E-TPGS, polyethylene glycol (PEG), ethoxylated hydrogenated castor oil (RH-40), Tween series surfactant and Span series surfactant. The carrier 20 includes at least one of cyclodextrin, maltodextrin, starch, silicon dioxide, cellulose, hydrogel and silicone hydrogel and is not limited to solid or liquid.

The present invention further provides a preparation method for the plant oil preparation as described above, including following steps of:

Step (1): mixing the at least one phospholipid material 12 in an aqueous phase 30 to form a dispersion solution. A weight ratio of the at least one phospholipid material 12 to the aqueous phase 30 is from 1:5 to 1:15 so as to provide a sufficient amount of the at least one phospholipid material 12 to facilitate encapsulation of the plant oil.

Step (2): mixing the plant oil in the dispersion solution to encapsulate at least a portion of the plant oil by the at least one phospholipid material 12 and form a plurality of intermediate particles 10a. A weight ratio of the plant oil to the aqueous phase 30 is from 1:4 to 1:8 so as to avoid excessive amount of the plant oil and control the particle size of the plurality of particles 10. In the step (2), the dispersion solution with the plant oil is stirred for no less than 20 minutes so that the plant oil is evenly dispersed within the dispersion solution to form a plurality of oil droplets, and the at least one phospholipid material 12 can attach to outer surfaces of the plurality of oil droplets via its lipophilic ends to form the plurality of intermediate particles 10a.

Step (3): adding the at least one surfactant 13, allowing the at least one surfactant 13 to attach to the plurality of intermediate particles 10a and form the plurality of particles 10. It is noted that, the at least one surfactant 13 must to be added after the plurality of intermediate particles 10a are stably formed to prevent the first protective layer 121 and the second protective layer 131 from mixing with each other into a single layer.

Step (4): adding the carrier 20 and dispersing the plurality of particles 10 within the carrier 20.

In the steps (1) to (4), a stirring speed maintains in a range of 100 rpm to 300 rpm so as to ensure that the plant oil, the plurality of intermediate particles 10a and the plurality of particles 10 are evenly dispersed and respectively mixed well with the at least one phospholipid material 12, the at least one surfactant 13 and the carrier 20. Preferably, the step (1) is performed at a temperature of 70° C. to 90° C. so that the at least one the phospholipid material 12 can be hydrated and dispersed in the aqueous phase 30; the steps (2) to (4) are performed at a temperature lower than 70° C. so as to prevent the particles from disintegration due to heat.

The following is a comparison between a preferable embodiment 1 and comparative examples 1 to 3. In this embodiment, the at least one phospholipid material 12 is lecithin; the plant oil is sea buckthorn fruit oil; the at least one surfactant 13 is Tween 80; and the carrier 20 is β-cyclodextrin. In the embodiment 1, a ratio of a sum of weights of lecithin and Tween 80 to a weight of sea buckthorn fruit oil is 5:2; and the comparative examples 1 to 3 are formulated with one of lecithin, Tween 80 and β-cyclodextrin in the same weight ratio as sea buckthorn fruit oil. In other words, in the comparative examples 1 and 2, a proportion of a weight of the plant oil to a sum of weights of the at least one phospholipid material 12 and the at least one surfactant 13 is unchanged. For preparing the plant oil preparation, 4.3 g lecithin is added to 43.2 ml water (the aqueous phase 30) and mixed well at 70° C., and 8.8 g sea buckthorn fruit oil is added thereto and stirred at 150 rpm for 20 minutes. Then, 17.5 g Tween 80 is added and mixed well to form the plurality of particles 10. Finally, 26.2 g β-cyclodextrin is added and mixed to form a slurry, which is the plant oil preparation. In this embodiment, step (5) is also included after the step (4): removing the aqueous phase 30 (for example, by freeze drying) to obtain the plant oil preparation being solidified, which is easy to store. The composition ratios of the embodiment 1 and the comparative examples 1 to 3 after solidification are listed in Table 1 below:

TABLE 1

| Composition | Embodi- | Comparative | Comparative | Comparative |
|---|---|---|---|---|
| (wt %) | ment 1 | example 1 | example 2 | example 3 |
| Sea buckthorn | 15.4 | 28.6 | 28.6 | 28.6 |
| fruit oil | | | | |
| Lecithin | 7.6 | 71.4 | — | — |
| Tween 80 | 30.8 | — | 71.4 | — |
| β-cyclodextrin | 46.2 | — | — | 71.4 |

The characterizations of the embodiment 1 and the comparative examples 1 to 3 are listed in Table 2 below. In table 2, “improvement of water solubility” and “improvement of antioxidant capacity” are ratios respectively based on the water solubility and the antioxidant capacity of pure sea buckthorn fruit oil; the antioxidant capacity is determined by DPPH radical scavenging assay. As shown in Table 2, the comparative example 3 is prepared without any emulsifier, which has no significant improvement to the water solubility of the plant oil; the comparative example 1 and the comparative example 2 improve the water solubility of the plant oil but have no significant improvement to the antioxidant capacity. With the first protective layer 121 and the second protective layer 131 formed by the at least one phospholipid material 12 and the at least one surfactant 13, the plant oil preparation has significant improvement to water solubility, and the encapsulation efficiency (EE) of the plant oil preparation is greater than 90%. In addition, compare with pure sea buckthorn fruit oil, the antioxidant capacity of the plant oil preparation is greatly increased by more than 10 times.

TABLE 2

| | Embodi- | Comparative | Comparative | Comparative |
|---|---|---|---|---|
| Item | ment 1 | example 1 | example 2 | example 3 |
| Improvement of | 39.9 | 38.1 | 15.7 | 0.17 |
| water solubility | | | | |
| Particle size (nm) | 242.5 | 203.1 | 198.6 | 423.7 |
| EE (%) | 91.2 | — | — | — |
| Improvement of | 11.2 | 1.27 | 1.32 | 1.07 |
| antioxidant | | | | |
| capacity | | | | |

Please refer to Table 3 below, a preferable embodiment 2 and comparative examples 4 to 6 are shown. The embodiment 2 is different from the embodiment 1 in that: the plant oil of the embodiment 2 is sea buckthorn seed oil. The types and amounts of the at least one phospholipid material 12, the at least one surfactant 13 and the aqueous phase 30 are the same as those in the embodiment 1.

TABLE 3

| Composition | Embodi- | Comparative | Comparative | Comparative |
|---|---|---|---|---|
| (wt %) | ment 2 | example 4 | example 5 | example 6 |
| Sea buckthorn | 15.4 | 28.6 | 28.6 | 28.6 |
| seed oil | | | | |
| Lecithin | 7.6 | 71.4 | — | — |
| Tween 80 | 30.8 | — | 71.4 | — |
| β-cyclodextrin | 46.2 | — | — | 71.4 |

The characterizations of the embodiment 2 and the comparative examples 4 to 6 are listed in Table 4 below. In table 4, “improvement of water solubility” and “improvement of antioxidant capacity” are ratios respectively based on the water solubility and the antioxidant capacity of pure sea buckthorn seed oil. As shown in Table 4, compare with the comparative examples 4 to 6, the preparation method of the present invention also can effectively improve the water solubility and the antioxidant capacity of sea buckthorn seed oil.

TABLE 4

| | Embodi- | Comparative | Comparative | Comparative |
|---|---|---|---|---|
| Item | ment 2 | example 4 | example 5 | example 6 |
| Improvement of | 114.2 | 46.4 | 29.5 | 0.63 |
| water solubility | | | | |
| Particle size (nm) | 309 | 305.9 | 124.8 | 407.0 |
| EE (%) | 92.3 | — | — | — |
| Improvement of | 10.6 | 1.69 | 1.59 | 1.30 |
| antioxidant | | | | |
| capacity | | | | |

Preferably, a step (6) is also included after the step (5): grinding the plant oil preparation being solidified to a size range of 40 mesh to 80 mesh to facilitate storage and subsequent applications (such as tableting or being added into lotions, etc.). The plant oil preparation being solidified may be ground by grinders or vibrating screens.

Although particular embodiments of the invention have been described in detail for purposes of illustration, various modifications and enhancements may be made without departing from the spirit and scope of the invention. Accordingly, the invention is not to be limited except as by the appended claims.

1. A plant oil preparation, including:
a plurality of particles, each of the plurality of particles including a core layer and at least one protective layer, the core layer including the plant oil, the at least one protective layer encapsulating the core layer and including at least one phospholipid material and at least one surfactant; and
a carrier, the plurality of particles being dispersed within the carrier.

2. The plant oil preparation of claim 1, wherein the at least one protective layer includes a first protective layer and a second protective layer, the at least one phospholipid material is disposed on an outer surface of the core layer to form the first protective layer, and the at least one surfactant is disposed on an outer surface of the first protective layer to form the second protective layer.

3. The plant oil preparation of claim 1, wherein a weight ratio of the at least one phospholipid material to the at least one surfactant is from 1:3 to 1:6.

4. The plant oil preparation claim 1, wherein the plant oil is present in an amount of at least 10% by weight of the plant oil preparation.

5. The plant oil preparation of claim 1, wherein an encapsulation efficiency of the plant oil is larger than or equal to 80%.

6. The plant oil preparation of claim 1, wherein an effective average particle size of the plurality of particles is between 150 nm and 400 nm.

7. The plant oil preparation of claim 1, wherein the plant oil includes at least one of sea buckthorn fruit oil, sea buckthorn seed oil, hemp oil and sacha inchi oil.

8. The plant oil preparation of claim 1, wherein the at least one phospholipid material includes at least one of soybean phospholipid, lecithin, dipalmitoylphosphatidylcholine (DPPC), distearoylphosphatidylcholine (DSPC) and egg phosphatidylcholine (EPC).

9. The plant oil preparation of claim 1, wherein the at least one surfactant includes at least one of vitamin E-TPGS, polyethylene glycol (PEG), ethoxylated hydrogenated castor oil (RH-40), Tween series surfactant and Span series surfactant.

10. The plant oil preparation of claim 1, wherein the carrier includes at least one of cyclodextrin, maltodextrin, starch, silicon dioxide, cellulose, hydrogel and silicone hydrogel.

11. A preparation method for the plant oil preparation of any claim 1, including following steps of:
(1) mixing the at least one phospholipid material in an aqueous phase to form a dispersion solution;
(2) mixing the plant oil in the dispersion solution to encapsulate the plant oil by the at least one phospholipid material and form a plurality of intermediate particles;
(3) adding the at least one surfactant, allowing the at least one surfactant to attach to the plurality of intermediate particles and form the plurality of particles; and
(4) adding the carrier and dispersing the plurality of particles within the carrier.

12. The preparation method of claim 11, wherein in the step (1), a weight ratio of the at least one phospholipid material to the aqueous phase is from 1:5 to 1:15.

13. The preparation method of claim 11, wherein in the step (2), a weight ratio of the plant oil to the aqueous phase is from 1:4 to 1:8.

14. The preparation method of claim 11, wherein the step (1) is performed at a temperature of 70° C. to 90° C., and the steps (2) to (4) are performed at a temperature lower than 70° C.

15. The preparation method of claim 11, wherein in the steps (1) to (4), a stirring speed maintains in a range of 100 rpm to 300 rpm.

16. The preparation method of claim 15, wherein in the step (2), the dispersion solution with the plant oil is stirred for no less than 20 minutes.

17. The preparation method of claim 11, wherein after the step (4), the aqueous phase is removed to obtain the plant oil preparation being solidified.

18. The preparation method of claim 17, further grinding the plant oil preparation being solidified to a size range of 40 mesh to 80 mesh.
